(19) United States
(12) Patent Application Publication
(10) Pub. No.: US 2016/0317770 A1
(43) Pub. Date: Nov. 3, 2016

(54) APNEA TREATMENT METHOD AND DEVICE

(71) Applicants: Clete Kushida, Los Altos Hills, CA (US); Roger S. Smith, Vernon, CT (US)

(72) Inventors: Clete Kushida, Los Altos Hills, CA (US); Roger S. Smith, Vernon, CT (US)

(21) Appl. No.: 15/209,684

(22) Filed: Jul. 13, 2016

Related U.S. Application Data
(63) Continuation of application No. 13/910,444, filed on Jun. 5, 2013, now abandoned.

(51) Int. Cl. A61M 16/06; A61M 16/00

(52) U.S. Cl. CPC A61M 16/0605; A61M 16/0057; A61M 16/0688; A61M 16/0694

(57) ABSTRACT

A closure for use during Positive Airway Pressure treatment of Obstructive Sleep Apnea for controlling air leakage through the lips and mouth of a person. The closure includes a first member formed of an air-sealing layer for extending over the lips of and to an adjacent region on the skin of the person. The closure includes a second member formed of an adhesive layer adhered to the first member for placement on at least the adjacent region on the skin for adhering the closure to the skin to form an air seal over the mouth. The closure includes a quick-release member for breaking the air seal.

### BACKGROUND OF THE INVENTION

This invention relates to method and apparatus for use in the treatment of obstructive sleep apnea (OSA) in people.

Obstructive sleep apnea occurs when the upper airway of a person becomes narrow or collapses during sleep. Such narrowing or collapsing, among other things, reduces oxygen in the blood and causes arousal from sleep. Obstructive sleep apnea is a chronic health problem that generally does not go away so that ongoing treatment is needed for a lifetime. Treatment for obstructive sleep apnea can be highly effective and often, the improvement in the quality of sleep and the quality of life will be noticed almost immediately. Not only do the quality of sleep and the quality of life improve for the person with obstructive sleep apnea, usually the person's sleep partner also benefits from markedly improved sleep quality and a reduction or elimination of the person's snoring.

Obstructive sleep apnea is a sleep disorder characterized by pauses (apneas) or decreases (hypopneas) in breathing occurring as a result of episodes of upper airway obstruction during sleep. Each episode during sleep causes one or more breaths to be missed. The episodes repeatedly occur during sleep. The episodes are frequently associated with oxyhemoglobin desaturations (3-4% or greater) and brief electroencephalographic arousals (a 3-second or greater shift in EEG frequency, measured at C3, C4, O1, or O2). Obstructive sleep apnea causes poor sleep, excessive daytime sleepiness, impaired mood and performance impairment such as increased risk of motor vehicle accidents. Obstructive sleep apnea is associated with hypertension, insulin resistance, cardiovascular disease including myocardial infarction and stroke. These results are considered to be related to the sleep disruption and/or the hypoxemia associated with the episodes of upper airway obstruction.

Obstructive sleep apnea occurs when a person's upper airway substantially narrows or closes during sleep. The physical anatomical structures of the airway are complex and are associated with the functioning of the air, food and fluid passages to enable breathing through the mouth and nose and to enable eating and drinking through the mouth. The food passages of the mouth, throat and esophagus leading to the stomach are separated from the airway by the soft palate and epiglottis and associated structures of the larynx to prevent food or fluid from passing into the airway and lungs.

The narrowing of the upper airway, that is the reduction in the luminal cross-sectional area of the upper airway, is a function of the net forces operating on the upper airway. The net forces are the algebraic sum of the closing forces (the forces tending to narrow or collapse the upper airway) and the opening forces (the forces tending to maintain or expand the upper airway). The closing forces include the extra-luminal tissue positive pressure force and intra-luminal negative pressure force occurring during inspiration. The opening forces include the intrinsic airway wall stiffness forces and the forces due to contraction of various airway dilator muscles. The net forces resulting from the interaction between the opening forces and the closing forces determines whether the upper airway remains open or collapsed. Sleep apnea occurs when the closing forces substantially exceed the opening forces.

Persons with obstructive sleep apnea during sleep do not tend to have obstructive apnea when awake. When awake, the upper airway tends to remain open due to increased activity of upper airway dilator muscles but this activity decreases during sleep predisposing the upper airway to narrow or to collapse. Anatomic narrowing from enlarged tonsils or tongue, peripharyngeal fat deposition, facial bony and upper airway structural characteristics, and other factors may also predispose the upper airway to more easily collapse under closing pressures during sleep. Other factors which contribute to obstructive sleep apnea include obesity, lack of fitness, aging, loss of general muscle tone, congestion of the throat and the effects of alcohol and smoking.

Treatments for obstructive sleep apnea usually rely upon Continuous Positive Airway Pressure (CPAP). Although there are other types of positive airway pressure devices (for example, Bilevel Airway Pressure (BPAP), Auto-Positive Airway Pressure (APAP), CPAP is the most commonly used. A CPAP device treats obstructive sleep apnea by delivering forced air through a nasal pillow, nose mask or a full-face mask to create a continuous positive airway pressure to form a “pneumatic splint” in the airway so that unobstructed breathing becomes possible and apneas and hypopneas are reduced or eliminated.

The continuous positive airway pressure is applied by a CPAP device to the upper airway to maintain the intra-luminal upper airway pressure above critical closing pressure at all times during the respiratory cycle. The presence of continuous positive airway pressure opposes the forces tending to narrow the upper airway and forms a “pneumatic splint” which holds the upper airway open. The term “pneumatic splint” describes the condition where the net forces operating on the upper airway hold the upper airway open and prevent narrowing or collapsing. The continuous positive airway pressure increases the intra-luminal upper airway cross-sectional area particularly in the lateral direction.

CPAP devices provide forced air at a prescribed pressure (called the titrated pressure) that is determined for each person. The titrated pressure is usually determined by a sleep physician during a sleep study (polysomnography) in a sleep laboratory. The titrated pressure for a person is the pressure of air at which apneas and hypopneas for that person have been substantially reduced or prevented. Titrated pressures are usually measured in centimeters of water (cm H₂O). The pressure required by most persons with sleep apnea ranges between 6 and 14 cm H₂O. A typical CPAP machine can deliver pressures between 4 and 20 cm H₂O and more specialized units can deliver pressures up to 30 cm H₂O.

The increase in intra-luminal upper airway cross-sectional area occurs in the presence of continuous positive airway pressure notwithstanding that the dilator muscle activity tends to be diminished when such positive pressure is applied. The presence of continuous positive airway pressure also tends to increase the upper airway size as a result of increased lung volume, particularly including increased lung volume at the end of expiration. The increase in upper airway size also is believed to occur as a result of physical wall stretching and attendant wall softening and/or dilation. For effective treatment of obstructive sleep apnea, it has been found that sufficient positive upper airway pressure must be maintained throughout the respiratory cycle. A collapse is most likely to occur at the end of expiration.

Nasal pillows, nasal masks and full-face masks (“CPAP interfaces”) deliver the air into the upper airway. In the example of a nasal mask, the mask is sealed over the nose to form a chamber including the mask and the nasal passages. The nasal mask includes an input port, typically connected through a hose to an air pump, for ingress of air under positive pressure from the pump. The positive pressure is applied to the upper airway during the whole respiratory cycle. The mask also includes an output port that allows egress of output air to the ambient air environment. The mask output port typically includes a restricted mask valve that controls the volume and output pressure of air flow from the mask to the ambient air environment.

The intended ideal operation of interfaces and methods is to control the upper airway to be in a “pneumatic splint” state through the application of continuous positive airway pressure. In such intended operation, the air flow into the mask through the input port equals the air flow out of the mask both through the output mask port and through the lower airways connected to the lungs. During inspiration for the intended ideal operation, some of the input air flow through the input port exits the mask as the inspiration airflow into the lower airways to the lungs while the remainder of the input air flow exits the mask through the mask output port. During expiration for the intended ideal operation, airflow from the lungs to the lower airways and airflow from the input port together exit the mask through the output mask port. With such intended ideal operation and when an outward air flow through the output mask port is maintained throughout the respiratory cycle, a positive air pressure in the mask and in the upper airway is maintained so as to establish the “pneumatic splint” condition that holds the upper airway open throughout the respiratory cycle.

Under the intended ideal operation described, the CPAP devices are intended to operate to facilitate unobstructed breathing so as to reduce or prevent apneas and hypopneas. While CPAP devices have been somewhat successful in achieving the intended operation, there still remain substantial problems to be overcome in CPAP devices and methods.

One significant problem with CPAP devices is “mouth leak”. When the mouth is open, competing air flow paths exist through the nose and through the mouth. For mouth breathing, air enters through the mouth, flows through the glottis (the opening from the pharynx into the larynx and into the trachea) into the respiratory tract and into the lungs. In the lungs, O₂ and CO₂ are exchanged and then the air flows back and exits through the mouth. When the mouth is closed, inspiration air flows from the ambient air through the nose into the respiratory tract and down into the lungs. In the lungs, O₂ and CO₂ are exchanged and then the expiration air flows back and exits through the nose.

For inspiration when a positive pressure air flow from a continuous positive airway pressure pump enters the nose in the upper airway, the air flow on inspiration is distributed along paths that balance the resistances to air flow. One inspiration air flow path is through the output port of the mask. Another inspiration air flow path is from the nose to the lower airways and into the lungs. Still another inspiration air flow path, when the mouth is open, is from the nose into the nasopharynx and out through the mouth. The inspiration air flow from the input port and the nose through the mouth is by way of the Vero lingual sphincter and the virtual oral cavity and out through the opening between lips. The inspiration and expiration air flow which passes through the mouth in a nasal CPAP environment is known as mouth leak. In a similar manner, the air flow on expiration is distributed along paths that balance the resistances to flow. When the mouth is open, the expiration air flow through the mouth may be substantial and under some conditions may prevent any air flow through the nose.

When such low or no air flow through the nose occurs during inspiration or expiration, the conditions that cause obstructive sleep apnea tend to be aggravated and hence the continuous positive air pressure treatment becomes uncomfortable and ineffective.

The effectiveness of CPAP devices is improved with a reduction or elimination of mouth leak. The use of CPAP devices with air leak frequently results in nasal symptoms including congestion, dry nose and throat, sore throat and bleeding nostrils.

People that breathe mainly through their mouths during sleep, compared to those who breathe mainly through their noses during sleep, have a greater tendency toward mouth leak when using CPAP devices.

Because people are less likely to continue use of the anti-apnea CPAP devices with mouth leak, and the resulting adverse symptoms of mouth leak, the effectiveness of such CPAP devices is materially reduced.

It has been estimated that sleep apnea effects from 2% to 10% of the population and sleep apnea becomes more common for older persons.

CPAP devices are the most commonly used treatment of sleep apnea. It is estimated that about 2.5 million CPAP devices are sold each year worldwide. The CPAP device treatment in general requires use each night while sleeping for a person's entire life. It is estimated that 40% of CPAP device users and almost 100% of BPAP device users experience mouth leak. In order to combat mouth leak in CPAP and BPAP devices, chin straps have been employed to keep the mouth closed and full face masks have been employed to cover both the mouth and the nose with continuous positive air pressure pushing into both. Both treatments are often not effective.

Persistent mouth leak most often means that the CPAP treatment is not effective ending in treatment failure where the mouth opens, air leaks, the pressure in the throat drops, the upper airway closes, apnea and snoring return, the mouth dries out and the patient pulls the CPAP mask off.

In consideration of the above background, there is a need for improved apparatus and methods for treatment of obstructive sleep apnea and particularly overcoming the problems of mouth leak.

### SUMMARY

The present invention is a closure for use during Positive Airway Pressure treatment of Obstructive Sleep Apnea for controlling air leakage through the lips and mouth of a person. The closure includes a first member formed of an air-sealing layer for extending over the lips of, and to an adjacent region on, the skin of the person. The closure includes a second member formed of an adhesive layer adhered to the first member for placement on at least the adjacent region on the skin for adhering the closure to the skin to form an air seal over the mouth. The closure includes a quick-release member for breaking the air seal.

In an embodiment, the quick-release member is a tab attached to the first member.

In an embodiment, the quick-release member is permeable member within a central region of the first member.

In an embodiment, the quick-release member is a central region of the first member where the central region is substantially thinner than other regions of the first member.

In an embodiment, the first member includes a chin strap.

In an embodiment, the second member is located around the periphery of the first member with an opening in the center so as not to contact the lips.

In an embodiment, the second member includes a first part located around the periphery of the first member and with a second part located within the first part where the second part has an opening in the center so as not to contact the lips.

In an embodiment, the second member includes a first part located around the periphery of the first member and with a second part located within the first part where the second part has an opening in the center so as not to contact the lips and wherein at least one the first part and the second part are removable to provide a closure with only the first part or the second part.

In an embodiment, the second member includes a part located within a first region located around the periphery of the first member where the second part has an opening in the center so as not to contact the lips.

In an embodiment, a set of closures are provided where each closure is for use during Positive Airway Pressure treatment of Obstructive Sleep Apnea for controlling air leakage through the lips and mouth of a person. Each closure in the set includes a first member formed of an air-sealing layer for extending over the lips of and to an adjacent region on the skin of the person. Each closure includes a second member formed of an adhesive layer adhered to the first member for placement on at least the adjacent region on the skin for adhering the closure to the skin to form an air seal over the mouth. Each second member for the set of closures is arrayed such that the placement on the skin occurs at different portions of the skin whereby the set of closures tend to reduce agitation of the skin. Each closure includes a quick-release member for breaking the air seal.

The foregoing and other objects, features and advantages of the invention will be apparent from the following detailed description in conjunction with the drawings.

### BRIEF DESCRIPTION OF THE DRAWINGS

FIG. 1 depicts a side-lying person with a mouth closure positioned to be moved over the mouth.

FIG. 2 depicts the side-lying person of FIG. 1 with a mouth closure, nose mask and air pump.

FIG. 3 depicts a top view of the mouth closure of FIG. 1 and FIG. 2.

FIG. 4 depicts a front edge view of the mouth closure of FIG. 3.

FIG. 5 depicts a back view of a first embodiment of the mouth closure of FIG. 3.

FIG. 6 depicts a back view of another embodiment of the mouth closure of FIG. 3.

FIG. 7 depicts a top view of another embodiment of a mouth closure having a penetrable membrane.

FIG. 8 depicts a front edge view of the mouth closure of FIG. 7.

FIG. 9 depicts a back view of the mouth closure of FIG. 7.

FIG. 10 depicts an end edge view of the mouth closure of FIG. 7 with the membrane perforated.

FIG. 11 depicts a front edge view of the mouth closure of FIG. 10.

FIG. 12 depicts a top view of another embodiment of a mouth closure.

FIG. 13 depicts a top view of another embodiment of a mouth closure.

FIG. 14 depicts a top view of another embodiment of a mouth closure.

FIG. 15 depicts a top view of another embodiment of a mouth closure.

FIG. 16 depicts a top view of another embodiment of a mouth closure.

FIG. 17 depicts a top view of another embodiment of a mouth closure similar to the mouth closure of FIG. 16.

FIG. 18 depicts a front edge view of the mouth closure of FIG. 17.

FIG. 19 depicts a back view of the mouth closure of FIG. 17.

FIG. 20 depicts a back view of another embodiment of a mouth closure.

FIG. 21 depicts a back view of the mouth closure of FIG. 19 and the mouth closure of FIG. 20 overlaid for comparison.

FIG. 22 depicts a top view of another embodiment of a mouth closure.

FIG. 23 depicts a top view of another embodiment of a mouth closure.

FIG. 24 depicts a top view of another embodiment of a mouth closure.

FIG. 25 depicts a top view of another embodiment of a mouth closure.

FIG. 26 depicts a top view of another embodiment of a mouth closure.

FIG. 27 depicts a back view of a first embodiment of the mouth closure of FIG. 26.

FIG. 28 depicts a back view of a second embodiment of the mouth closure of FIG. 26.

FIG. 29 depicts a top view of another embodiment of a mouth closure.

FIG. 30 depicts a back view of the mouth closure of FIG. 29.

FIG. 31 depicts a top view of another embodiment of a mouth closure.

FIG. 32 depicts a back view of the mouth closure of FIG. 31.

FIG. 33 depicts a top view of another embodiment of a mouth closure.

FIG. 34 depicts a back view of the mouth closure of FIG. 33.

FIG. 35 depicts a cross-sectional anatomical view, in the back-lying position, of a person with the uncovered mouth closed and breathing through the nose.

FIG. 36 depicts a cross-sectional anatomical view, in the back-lying position of a person with the uncovered mouth open and with breathing through the nose and the mouth.

FIG. 37 depicts a cross-sectional anatomical view, in the back-lying position with the uncovered mouth open, of a person with obstructed breathing air flow through the nose and mouth.

FIG. 38 depicts a cross-sectional anatomical view, in the back-lying position with the uncovered mouth open, of a person with a positive pressure nose mask and with obstructed breathing air flow leakage through the mouth.

FIG. 39 depicts a cross-sectional anatomical view, in the back-lying position with the mouth closed and sealed by a mouth closure, of a person with a positive pressure nose mask and with obstructed breathing air flow facilitated without leakage through the mouth.

FIG. 40 depicts a cross-sectional anatomical view, in the back-lying position with the mouth closed, of the person of FIG. 24 with a positive pressure nose mask and the mouth closure perforated allowing air leakage through the mouth.

### DETAILED DESCRIPTION

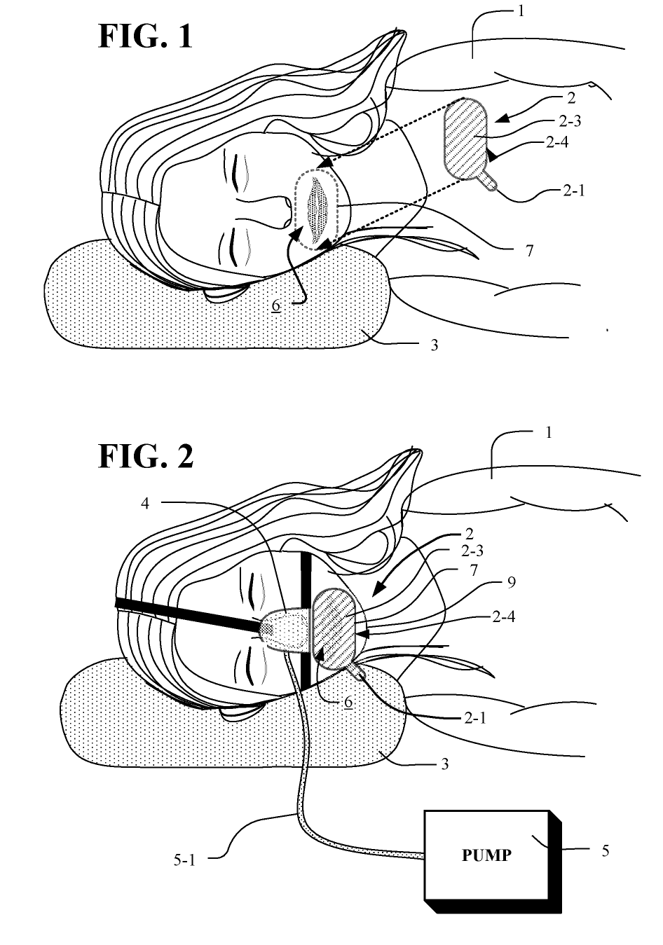

In FIG. 1, a side-lying person 1 is reclining on pillow 3. A closure 2 is provided for placement over the lips 6 for sealing the mouth opening. When the mouth and lips are closed, the closure 2 is placed over the lips 6 and the adjacent skin region 7 and is affixed to form a seal to prevent or reduce mouth breathing. The closure 2 includes a first member 2-3 formed of an air-sealing layer for extending over the lips 6 and to the adjacent region 7 on the skin of the person 1. The closure 2 includes a second member formed of an adhesive layer 2-4 under and adhered to the first member 2-3 for placement in at least the adjacent region 7 of the skin for adhering the closure 2 to the skin to form an air seal over the mouth. The first member 2-3 and second member 2-4 cover the whole mouth without any uncovered regions so as to prevent air leakage through the mouth.

The closure 2, when adhered to the skin by the adhesive member 2-4, forms a seal over the lips 6 and hence seals the mouth to prevent or reduce mouth air leak during Positive Airway Pressure treatment of Obstructive Sleep Apnea. The closure 2 includes a quick-release member 2-1 which is used for breaking the air seal over the mouth. The quick-release member, in the form of a tab 2-1 in FIG. 1, is grasped and pulled for releasing the seal of the closure 2 and allowing air to flow through the mouth and the lips 6. The quick-release member 2-1 is a safety feature that allows a person, such as person 1, to quickly release the seal of the closure 2 and quickly allow breathing through the mouth without necessarily having to completely remove the closure 2 from the person 1.

In FIG. 2, the side-lying person 1 of FIG. 1 is reclining on pillow 3 with a mouth closure 2, a nose mask 4 and an air pump 5. The pump 5 pumps air between the mask 4 and the pump 5 through the connecting tube 5-1 to establish Continuous Positive Airway Pressure (CPAP) for the person 1. During inspiration the pump 5 increases the air flow output to compensate for the airflow leaving the mask-upper airway chamber and entering the lower airways and lungs. During expiration, the pump decreases its output since air is also entering the mask-upper airway chamber from the lungs/lower airways.

In FIG. 2, the closure 2 includes a first member 2-3 formed of an air-sealing layer for extending over the lips 6 and to the adjacent region 7 on the skin of the person 1. The closure 2 includes a second member formed of an adhesive layer 2-4 adhered to the first member 2-3 for placement on at least the adjacent region 7 of the skin for adhering the closure 2 to the skin to form an air seal over the mouth. The closure 2, when adhered to the skin by the adhesive member 2-4, forms a seal over the lips 6 and hence seals the mouth to prevent or reduce mouth air leak during Positive Airway Pressure treatment of Obstructive Sleep Apnea. The closure 2 includes a quick-release member 2-1 which is used for breaking the air seal over the mouth. The quick-release member, in the form of a tab 2-1 in FIG. 1, is grasped and pulled for releasing the seal of the closure 2 and allowing air to flow through the mouth and the lips 6. The quick-release member 2-1 is a safety feature that allows a person, such as person 1, to quickly release the seal of the closure 2 and quickly allow breathing through the mouth without necessarily having to completely remove the closure 2 from the person 1.

The closure 2 typically has the properties of being comfortable, hypoallergenic, waterproof, breathable and capable of being easily removed to quickly allow the seal to be broken and allow air to pass through the lips. Examples of medical/surgical tapes suitable for making mouth closures are as follows.

The 3M™ 2962 Medipore™ Soft Cloth Surgical Tape, available from the 3M Company, is a non-woven polyester, soft, conformable, porous and breathable tape that is useful for repeated applications on skin and which stretches to accommodate swelling and movement without curling edges and without sticking to itself.

The HyTape® (HYT105BLF) tape, available from Hy-Tape® International, is waterproof, washable, latex-free, zinc oxide-based, soothing and delicate skin and which can be applied to wet skin without slipping or detaching and which conforms easily to body contours.

The Kendall 6613 Curity™ Standard Porous Tape, available from the Kendall Co (Covidien), has linear porosity for high breathability, high tensile strength, strong adhesion.

The 3M™ 15302 Micropore™ Surgical Paper Tape available from the 3M Company is breathable, conformable, hypoallergenic, and suitable for repeated applications on sensitive skin and which allows for moisture evaporation and skin breathing with minimal adhesive residue upon removal. The 3M™ 1521, 1525, 9865A, 9832, 9833 medical tapes are also suitable for making closures.

The Omnifix Dressing Retention Tape, available from the Conco Medical Company, is a latex-free, soft, non-woven, acrylic, wave slit paper backed which is air and water vapor permeable.

The Molnlycke Mepitac Soft Silicone Tape, available from the Molnlycke Health Care Company, is made of a soft silicone contact layer, a knitted fabric, and a breathable polyurethane film.

The Zonas Porous Tape, available from Johnson & Johnson, is also known as athletic tape, or sports tape, and has porous construction with a rubber base adhesive, cotton cloth backing and low unwind tension.

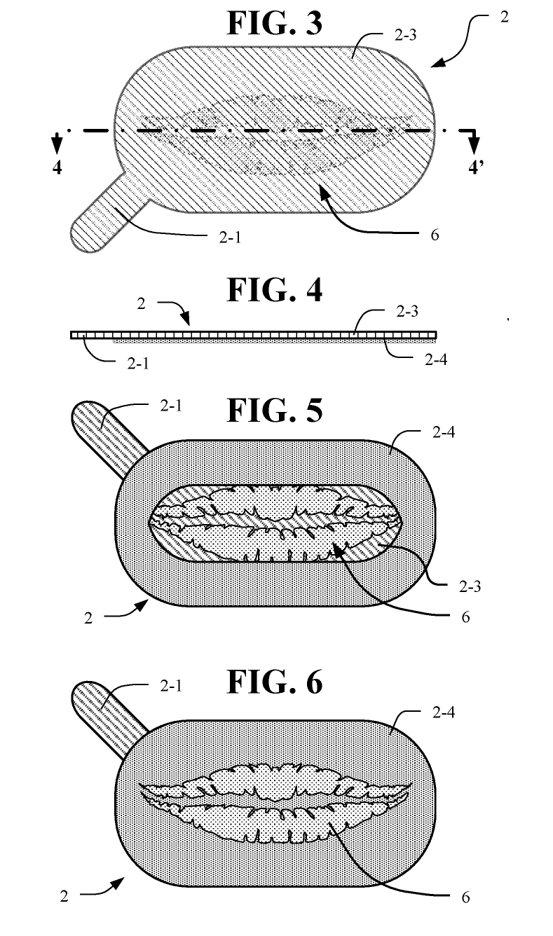

In FIG. 3, a top view is shown of the mouth closure 2 of FIG. 1 and FIG. 2. The closure 2 includes an air-sealing first layer 2-3. The closure 2 is typically from 1.5 to 3.5 inches in width and from 0.5 to 1.5 inches high. Although these dimensions are typical, larger and smaller dimensions also are acceptable since there is a large variation among the general population in lip and mouth sizes. The objective is to cover the lips and to extend beyond the lips with an adhesive border in order to seal the mouth. An image of the lips 6 is shown to indicate the relative size of the closure 2. Typically, a high percentage of the general population lip and mouth sizes can be provided for with closure sizes of small, medium and large with the sizes ranging from 1.5 to 3.5 inches in width and from 0.5 to 1.5 inches high. Some closures may need to be larger, for larger lips and mouths or for a larger adhesion region beyond the lips. The larger sizes can extend from 6 to 8 inches or more. The closure 2 includes a quick-release member in the form of tab 2-1 that is available for grasping, for example by the person 1 of FIG. 1 and FIG. 2, for releasing the adhesive seal of the closure 2 to allow air to pass through the lips 6 and through the mouth of the person.

In FIG. 4, a front edge view of the mouth closure 2 of FIG. 3 is shown taken along the section line 4-4′ of FIG. 3. The closure 2 includes an air-sealing first layer 2-3 and an air-sealing second adhesive layer 2-4 that seals the first layer 2-3 around and over the lips 6 as shown in FIG. 3. The quick-release member in the form of tab 2-1 does not have any adhesive and therefore is readily grasped for pulling and quick release of the seal formed by the adhesive layer 2-4 of the mouth closure 2.

In FIG. 5, a back view of one embodiment of the mouth closure 2 of FIG. 3 is shown. The closure 2 includes an air-sealing first layer 2-3 and an air-sealing second adhesive layer 2-4 that seals the first layer 2-3 around and over the lips 6 of FIG. 3. The adhesive layer 2-4 is located around the outer edge of the air-sealing first layer 2-3 and hence the adhesive layer 2-4, when adhered to a person 1 as shown in FIG. 2, surrounds the lips 6 without substantial contact to the lips 6. The adhesive layer 2-4 is shaped as a rounded rectangle with adhesive having a width placed around the periphery of the first layer 2-3 leaving a hole in the center so as not to contact the lips 6. The first member 2-3 and second member 2-4 cover the whole mouth without any uncovered regions so as to prevent air leakage through the mouth.

The width of the outer dimension is typically between 0.25 inch and 0.75 inch and the hole is typically 0.5 inch to 1 inch wide and 1.5 inches to 3 inches long. The dimensions are only by way of example since the objective in FIG. 5 is to cover the lips, without substantial adhesive contact to the lips, and to extend beyond the lips with an adhesive border. The quick-release member in the form of tab 2-1 does not have any adhesive and therefore is readily grasped for pulling and quick release of the seal formed by the adhesive layer 2-4 of the mouth closure 2.

In FIG. 6, a back view of another embodiment of the mouth closure 2 of FIG. 3 is shown. The closure 2 includes an air-sealing second adhesive layer 2-4 (an air-sealing first layer 2-3 (over an air-sealing first layer, not shown, see FIG. 3 and FIG. 4) that seals the first layer 2-3 over the lips 6 and surrounding skin of FIG. 3. The adhesive layer 2-4 is located over the air-sealing first layer 2-3 and hence the adhesive layer 2-4, when adhered to a person 1 as shown in FIG. 2, contacts the lips 6 and the skin surrounding the lips 6. The dimensions of the closure 2 range from 1.5 to 3.5 inches in width and from 0.5 to 1.5 inches high. Some of the closures may need to be larger, for larger lips and mouth or larger adhesion area, extending to as much as from 6 to 8 inches or more. The dimensions are only by way of example since the objective in FIG. 6 is to cover the lips, including adhesive contact to the lips, and to extend beyond the lips with an adhesive border. The quick-release member in the form of tab 2-1 does not have any adhesive and therefore is readily grasped for pulling and quick release of the seal formed by the adhesive layer 2-4 of the mouth closure 2.

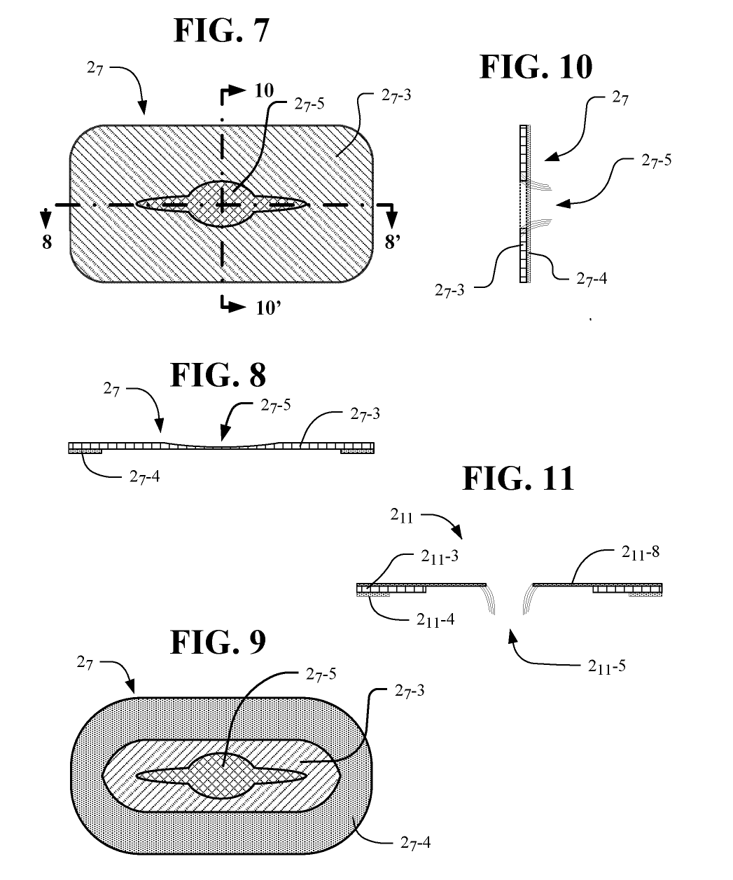

In FIG. 7, a top view of another embodiment of a mouth closure 27 having a penetrable membrane 2₇-5. The closure 27 includes an air-sealing first layer 2₇-3. The closure 2₇ is typically from 1.5 to 3.5 inches in width and from 0.5 to 1.5 inches high and larger and smaller dimensions also are acceptable. The penetrable membrane 2₇-5 covers a portion of the air-sealing first layer 2₇-3 and until penetrated is air-sealing. When a person, such as person 1 in FIG. 2, has an urgent need for air through the mouth, a finger can be used to easily puncture the membrane 2₇-5 to allow air to pass through the lips 6 and the mouth.

In FIG. 8, a front edge view of the mouth closure 2₇ of FIG. 7 is shown taken along the section line 8-8′ of FIG. 7. The closure 2₇ includes an air-sealing first layer 2₇-3 and an air-sealing second adhesive layer 2₇-4 that seals the first layer 2₇-3 around and over the lips 6 in a similar manner as shown in FIG. 3. The quick-release member in the form of the penetrable membrane 2₇-5, in the example of FIG. 8, is a substantial thinning of the first layer 2₇-3 in the membrane 2₇-5 region. The membrane 2₇-5 region is a central region of the first layer 2₇-3 where the central region is substantially thinner than other regions of the first layer 2₇-3. Alternatively, the region of the penetrable membrane 2₇-5 can be a different material than first layer 2₇-3 where the different material has greater permeability than the material of the first layer 2₇-3. Still further, the region of the penetrable membrane 2₇-5 can be perforated to allow easy penetration.

In FIG. 9, a back view of the mouth closure 2₇ of FIG. 7 is shown. The closure 27 includes an air-sealing first layer 2₇-3 and an air-sealing second adhesive layer 2₇-4 that seals the first layer 2₇-3 around and over the lips 6 in a similar manner as shown in FIG. 3. The adhesive layer 2₇-4 is located around the outer edge of the air-sealing first layer 2₇-3 and hence the adhesive layer 2₇-4, when adhered to a person 1 as shown in FIG. 2, surrounds the lips 6 without substantial contact to the lips 6. The adhesive layer 2₇-4 is shaped as a rounded rectangle with adhesive having a width placed around the periphery of the first layer 2₇-3 leaving a hole in the center so as not to contact the lips 6. The dimensions of the closure 2₇ are similar to those described in connection with the closure 2 of FIG. 5. The objective in FIG. 9 is to cover the lips, without substantial adhesive contact to the lips, and to extend beyond the lips with an adhesive border. The quick-release member in the form of the penetrable membrane 2₇-5 is in the center region of the layer 2₇-3 without any adhesive. The seal is readily broken by using a finger to penetrate the penetrable membrane 2₇-5.

In FIG. 10, an end edge view of the mouth closure 2₇ of FIG. 7 is shown taken along the section line 10-10′ of FIG. 7. The closure 2₇ includes an air-sealing first layer 2₇-3 and a second adhesive layer 2₇-4. The first layer 2₇-3 includes penetrable membrane 2₇-5 that has been punctured to break the seal of the first layer 2₇-3.

In FIG. 11, a front edge view of another embodiment, closure 2₁₁, of the mouth closure of FIG. 7 is shown. The closure 2₁₁ view is taken along the section line 8-8′ of FIG. 7. The FIG. 11 embodiment includes an air-sealing first layer 2₁₁-3, an air-sealing second adhesive layer 2₁₁-4 and an air-sealing third layer 2₁₁-8. The third layer 2₁₁-8 covers the first layer 2₁₁-3. The first layer 2₁₁-3 in one typical example has an opening shaped like the region 2₇-5 in FIG. 9 which when not punctured is covered by the third layer 2₁₁-8. Together, the first layer 2₁₁-3, the second layer 2₁₁-4 and the third layer 2₁₁-8 form a seal over the lips and the mouth of a person, such as person 1 in FIG. 2. In FIG. 11, the third layer 2₁₁-8 is shown punctured at 2₁₁-5 so that the seal over the mouth is broken.

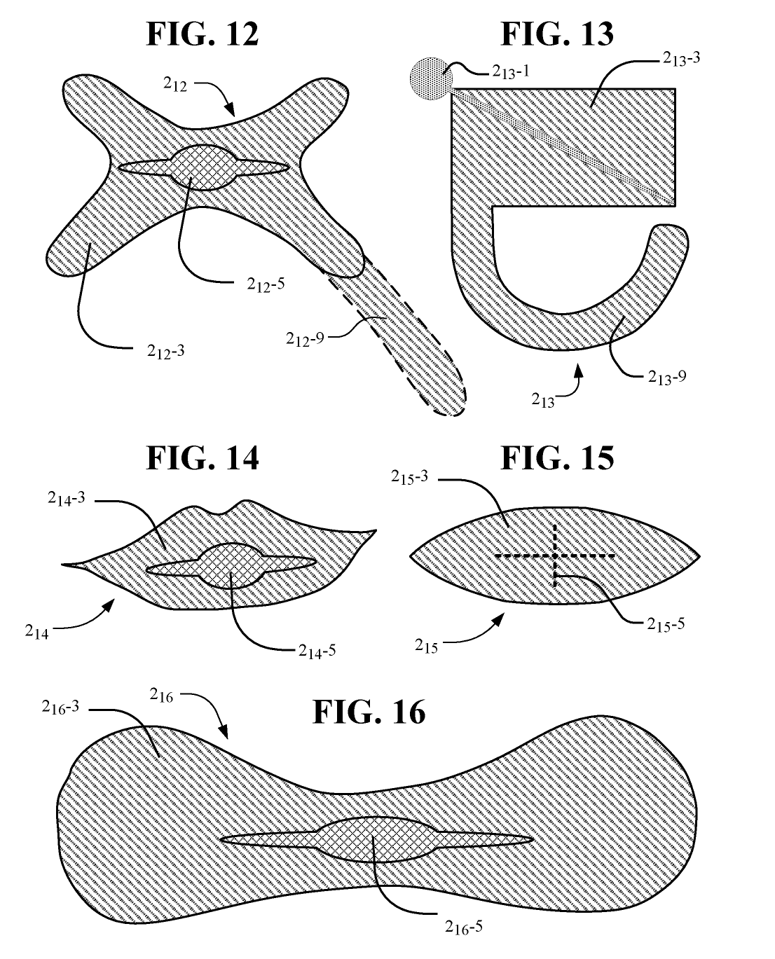

In FIG. 12, a top view of another embodiment of a mouth closure 2₁₂ is shown. The closure 2₁₂ includes an air-sealing first layer 2₁₂-3 over a second adhesive layer (not shown, but like the layer 2-4 in FIG. 4 or FIG. 5). The closure 2₁₂ includes a penetrable membrane 2₁₂-5 that is easily punctured by a person to break the mouth air seal. The closure 2₁₂ includes, in an alternate embodiment, a chin strap 2₁₂-9 which functions to extend under a chin, for example chin 9 of the person 1 of FIG. 2. The chin strap 2₁₂-9 functions to help hold the mouth closed of a person, for example person 1 in FIG. 2, when the mouth is sealed by the closure 2₁₂.

In FIG. 13, a top view of another embodiment of a mouth closure 2₁₃ is shown. The closure 2₁₃ includes an air-sealing first layer 2₁₃-3 over a second adhesive layer (not shown, like the layer 2-4 in FIG. 4 or FIG. 5). The closure 2₁₃ includes a quick-release member in the form of tab 2₁₃-1 that does not have any adhesive and therefore is readily grasped for pulling and quick release of the seal formed by the closure 2₁₃. The closure 2₁₃ includes a chin strap 2₁₃-9 which functions to extend under a chin, for example chin 9 of the person 1 of FIG. 2. The chin strap 2₁₃-9 functions to help hold the mouth closed of a person, for example person 1 in FIG. 2, when the mouth is sealed by the closure 2₁₃.

In FIG. 14, a top view of another embodiment of a mouth closure 2₁₄ is shown. The closure 2₁₄ includes an air-sealing first layer 2₁₄-3 over a second adhesive layer (not shown, like the layer 2-4 in FIG. 4 or FIG. 5). The closure 214 includes a quick-release member in the form of a penetrable membrane 2₁₄-5 in a portion of the air-sealing first layer 2₁₄-3 and until penetrated the membrane 2₁₄-5 is air-sealing. When a person, such as person 1 in FIG. 2, has an urgent need for air through the mouth, a finger can be used to easily puncture the membrane 2₁₄-5 to allow air to pass through the lips 6 and the mouth.

In FIG. 15, a top view of another embodiment of a mouth closure 2₁₅ is shown. The closure 2₁₅ includes an air-sealing first layer 2₁₄-3 over a second adhesive layer (not shown, like the layer 2-4 in FIG. 4 or FIG. 5). The closure 2₁₅ includes a quick-release member in the form of crossed scores 2₁₅-5 in a portion of the air-sealing first layer 2₁₄-3 and until penetrated the crossed scores 2₁₅-5 are air-sealing. When a person, such as person 1 in FIG. 2, has an urgent need for air through the mouth, a finger can be used to easily puncture the membrane 2₁₅-3 by pressing a finger through at the crossed scores 2₁₅-5 to allow air to pass through the lips 6 and the mouth.

In FIG. 16, a top view of another embodiment of a mouth closure 2₁₆ is shown. The air sealing and adhesive layers extend further laterally than the other embodiments to help reduce billowing of the cheeks upon expiration while using PAP. The closure 2₁₆ includes an air-sealing first layer 2₁₆-3 over a second adhesive layer (not shown, like the layer 2-4 in FIG. 4 or FIG. 5). The closure 2₁₆ includes a quick-release member in the form of a penetrable membrane 2₁₆-5 in a portion of the air-sealing first layer 2₁₄-3 and until penetrated, the membrane 2₁₆-5 is air-sealing. When a person, such as person 1 in FIG. 2, has an urgent need for air through the mouth, a finger can be used to easily puncture the membrane 2₁₆-5 to allow air to pass through the lips 6 and the mouth.

In FIG. 17, a top view of another embodiment of a mouth closure 2₁₇ is shown. The closure 2₁₇ includes an air-sealing first layer 2₁₇-3 over a second adhesive layer (not shown, like the layer 2-4 in FIG. 4 or FIG. 5). The closure 2₁₇ includes a quick-release member in the form of tab 2₁₇-1 that does not have any adhesive and therefore is readily grasped for pulling and quick release of the seal formed by the closure 2₁₇.

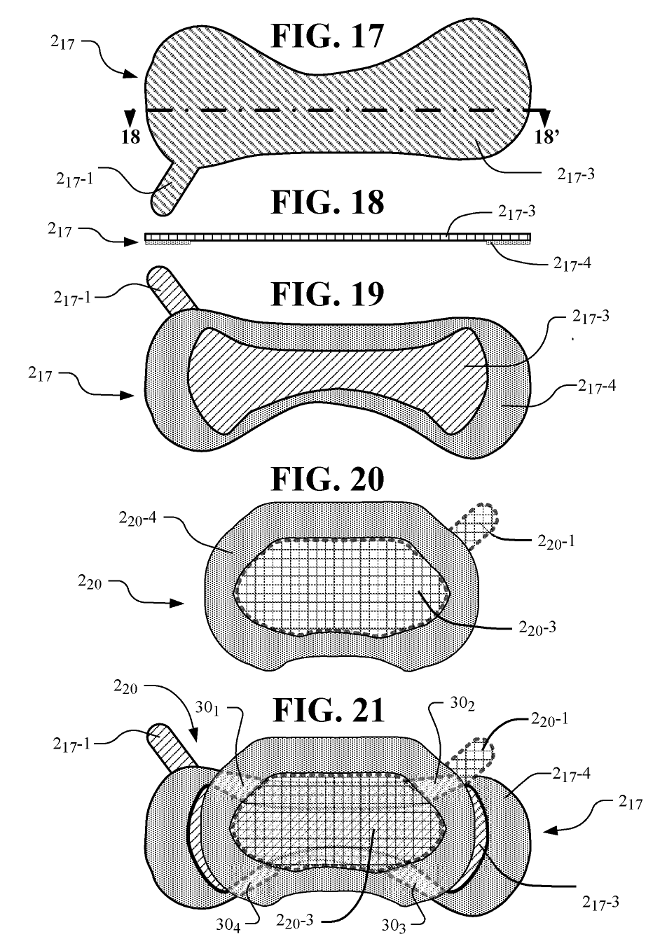

In FIG. 18, a front edge view of the mouth closure 2₁₇ of FIG. 17 is shown taken along the section line 18-18′ of FIG. 17. The closure 2 includes an air-sealing first layer 2₁₇-3 and an air-sealing second adhesive layer 2₁₇-4 that seals the first layer 2₁₇-3 around and over the lips 6 in a manner similar to FIG. 3. The quick-release member in the form of tab 2₁₇-1 does not have any adhesive and therefore is readily grasped for pulling and quick release of the seal formed by the adhesive layer 2₁₇-4 of the mouth closure 2₁₇.

In FIG. 19, a back view of the mouth closure 2₁₇ of FIG. 17 is shown. The closure 2₁₇ includes an air-sealing first layer 2₁₇-3 and an air-sealing second adhesive layer 2₁₇-4 that seals the first layer 2₁₇-3 around and over the lips 6 in a manner similar to FIG. 3. The adhesive layer 2₁₇-4 is located around the outer edge of the air-sealing first layer 2₁₇-3 and hence the adhesive layer 2₁₇-4, when adhered to a person 1 as shown in FIG. 2, surrounds the lips 6 without substantial contact to the lips 6. The adhesive layer 2₁₇-4 is shaped as a bulged rectangle with adhesive having a width placed around the periphery of the first layer 2₁₇-3 leaving an opening in the center so as not to contact the lips 6. The objective in FIG. 19 is to cover the lips, without substantial adhesive contact to the lips, and to extend beyond the lips with an adhesive border. The quick-release member in the form of tab 2₁₇-1 does not have any adhesive and therefore is readily grasped for pulling and quick release of the seal formed by the adhesive layer 2₁₇-4 of the mouth closure 2₁₇.

In FIG. 20, a back view of a mouth closure 2₂₀ is shown. The closure 2₂₀ includes an air-sealing first layer 2₂₀-3 and an air-sealing second adhesive layer 2₂₀-4 that seals the first layer 2₂₀-3 around and over the lips 6 in a manner similar to FIG. 3. The adhesive layer 2₂₀-4 is located around the outer edge of the air-sealing first layer 2₂₀-3 and hence the adhesive layer 2₂₀-4, when adhered to a person 1 as shown in FIG. 2, surrounds the lips 6 without substantial contact to the lips 6. The quick-release member in the form of tab 2₂₀-1 does not have any adhesive and therefore is readily grasped for pulling and quick release of the seal formed by the adhesive layer 2₂₀-4 of the mouth closure 2₂₀. The closure 2₂₀ is shaped to complement the shape of the closure 2₁₇ in FIG. 19. The adhesive layer 2₂₀-4 has a different shape than the adhesive layer 2₁₇-4. A person wearing the closure 2₂₀ on one day and wearing the closure 2₁₇ on another day will have a small overlap of adhesive layer on the skin over the two days as seen in connection with FIG. 21.

FIG. 21 depicts a back view of the closure 2₁₇ of FIG. 19 and the closure 2₂₀ of FIG. 20 superimposed for comparison of the adhesive layers as they would be projected on the skin of a wearer. The only regions that are common between the adhesive layer 2₂₀-4 and the adhesive layer 2₁₇-4 are the small regions 30₁, 30₂, 30₃ and 30₄. The closure 2₁₇ and the closure 2₂₀ are examples of closures that have a small overlap of adhesive layer on the skin. Many different shapes may be employed to form sets of closures that have small or no overlaps so that rotation from day to day reduces skin irritation.

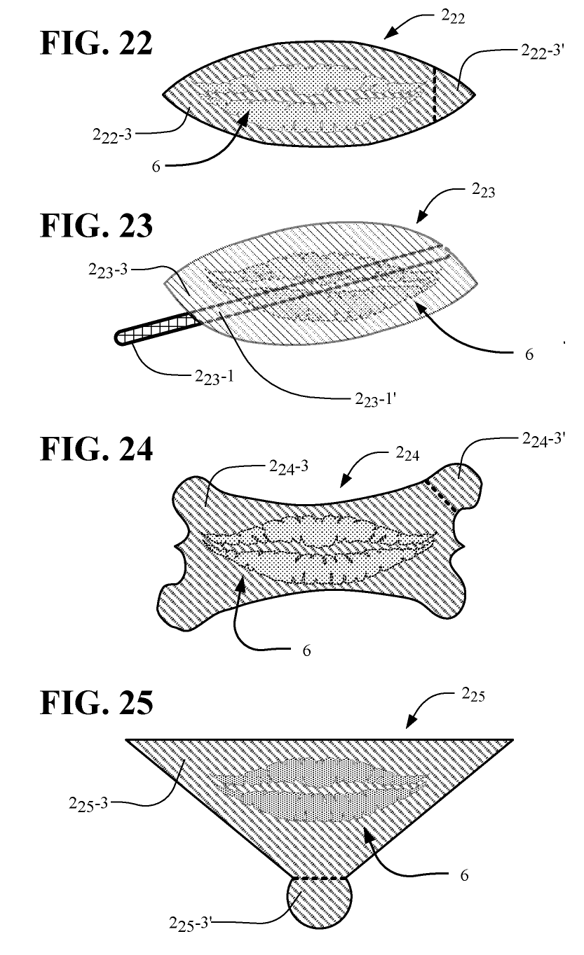

In FIG. 22, a top view of another embodiment of a mouth closure 2₂₂ is shown. The closure 2₂₂ includes an air-sealing first layer 2₂₂-3 over a second adhesive layer (not shown, but like the layer 2-4 in FIG. 4 or FIG. 5). The closure 2₂₂ includes a quick-release member in the form of tab 2₂₂-3′, portion of the first layer 2₂₂-3. The tab 2₂₂-3′ does not have any adhesive and therefore is readily grasped for pulling and quick release of the seal formed by the adhesive layer, similar to the adhesive layer 2-4 in FIG. 4 or FIG. 5.

In FIG. 23, a top view of another embodiment of a mouth closure 2₂₃ is shown. The closure 2₂₃ includes an air-sealing first layer 2₂₃-3 over a second adhesive layer (not shown, like the layer 2-4 in FIG. 4 or FIG. 5). The closure 2₂₃ includes a quick-release member in the form of tab 2₂₃-1 connected to a rip section 2₂₃-1′. The tab 2₂₃-1 does not have any adhesive and therefore is readily grasped for pulling and quick release of the seal formed by the closure 2₂₃. The rip section 2₂₃-1 when pulled tears the rip section 2₂₃-1′ to break the seal.

In FIG. 24, a top view of another embodiment of a mouth closure 2₂₄ is shown. The closure 2₂₄ includes an air-sealing first layer 2₂₄-3 over a second adhesive layer (not shown, but like the layer 2-4 in FIG. 4 or FIG. 5). The closure 2₂₄ includes a quick-release member in the form of tab 2₂₄-3′ which is attached to first layer 2₂₃-3. The tab 2₂₃-3′ does not have any adhesive and therefore is readily grasped for pulling and quick release of the seal formed by the adhesive layer, similar to the adhesive layer 2-4 in FIG. 4 or FIG. 5.

In FIG. 25, a top view of another embodiment of a mouth closure 2₂₅ is shown.

The closure 2₂₅ includes an air-sealing first layer 2₂₅-3 over a second adhesive layer (not shown, but like the layer 2-4 in FIG. 4 or FIG. 5). The closure 2₂₅ includes a quick-release member in the form of tab 2₂₅-3′ which is attached to first layer 2₂₅-3. The tab 2₂₅-3′ does not have any adhesive and therefore is readily grasped for pulling and quick release of the seal formed by the adhesive layer, similar to the adhesive layer 2-4 in FIG. 4 or FIG. 5.

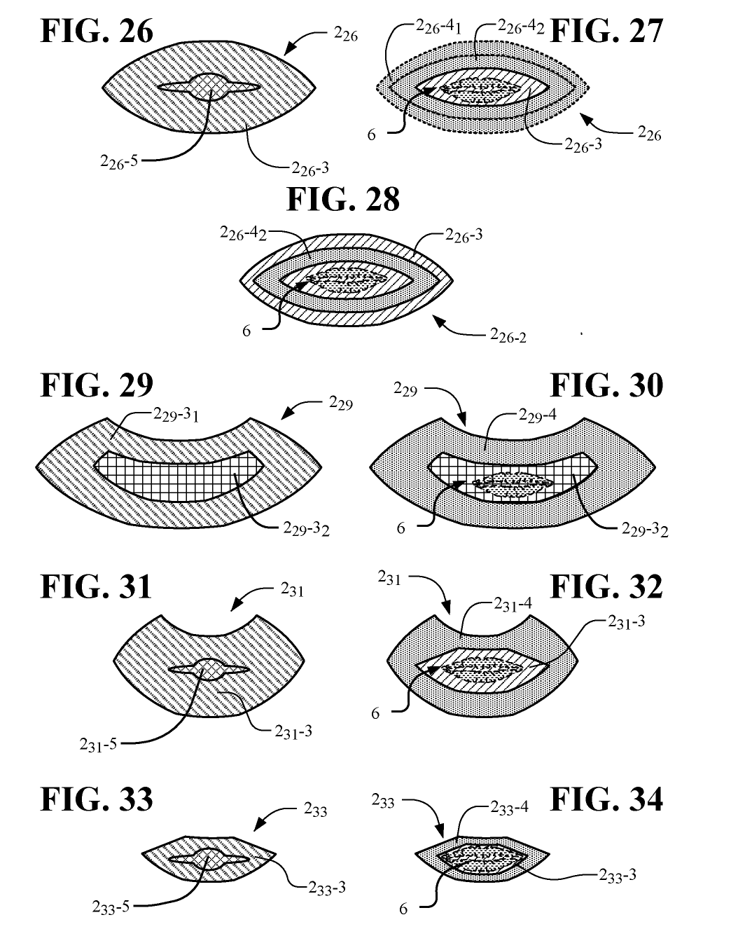

In FIG. 26, a top view of another embodiment of a mouth closure 2₂₆ is shown. The closure 2₂₆ includes an air-sealing first layer 2₂₆-3 over a second adhesive layer (not shown, but like the layer 2-4 in FIG. 4 or FIG. 5). The closure 2₂₆ includes a quick-release member in the form of a penetrable membrane 2₂₆-5 in a portion of the air-sealing first layer 2₂₆-3 and until penetrated the membrane 2₂₆-5 is air-sealing. When a person, such as person 1 in FIG. 2, has an urgent need for air through the mouth, a finger can be used to easily puncture the membrane 2₂₆-5 to allow air to pass through the lips 6 and the mouth.

In FIG. 27, a back view of a first embodiment of the mouth closure 2₂₆ of FIG. 26 is shown. The closure 2₂₆ includes an air-sealing first layer 2₂₆-3 and an air-sealing second adhesive layer 2₂₆-4₁ that seals the first layer 2₂₆-3 around and over the lips 6 in a manner similar to FIG. 3. The adhesive layer 2₂₆-4₁ is located around the outer edge of the air-sealing first layer 2₂₆-3 and hence the adhesive layer 2₂₆-4₁, when adhered to a person 1 as shown in FIG. 2, surrounds the lips 6 without substantial contact to the lips 6. The adhesive layer 2₂₆-4₁ is shaped having a width placed around the periphery of the first layer 2₂₆-3 leaving an opening in the center. The first layer 2₂₆-3 includes a quick-release member in the form of the membrane 2₂₆-5.

In FIG. 28, a back view of a second embodiment of the mouth closure 2₂₆ of FIG. 26 is shown. The closure 2₂₆ includes an air-sealing first layer 2₂₆-3 and an air-sealing second adhesive layer 2₂₆-4₂ that seals the first layer 2₂₆-3 around and over the lips 6 in a manner similar to FIG. 3. The adhesive layer 2₂₆-4₂ is located so as not to extend to the outer edge of the air-sealing first layer 2₂₆-3 while at the same time the adhesive layer 2₂₆-4₂, when adhered to a person 1 as shown in FIG. 2, surrounds the lips 6 without substantial contact to the lips 6. The adhesive layer 2₂₆-4₂ is shaped to be offset from the adhesive layer 2₂₆-4₁ of FIG. 27. The closure 2₂₆ with the first embodiment adhesive layer 2₂₆-4₁ and the second adhesive layer 2₂₆-4₂ are examples of closures that have a no overlap of adhesive layer on the skin when used at different times. The adhesive layer 2₂₆-4₁ is located around the periphery of the first layer 2₂₆-3 and the second adhesive layer 2₂₆-4₂ fits entirely within the projection of the adhesive layer 2₂₆-4₁ on the skin. Many different shapes may be employed to form sets of closures that have small or no overlaps so that use of different ones from day to day reduces skin irritation.

Referring to FIG. 27 and FIG. 28, the first and second embodiments of the closure 2₂₆ are alternatively combined in a third embodiment. In the third embodiment, the adhesive layer 2₂₆-4₁ and the second adhesive layer 2₂₆-4₂ are combined on the same first layer 2₂₆-₃ with a score between then. A user therefore is able to use the closure 2₂₆ with both the adhesive layer 2₂₆-4₁ and the second adhesive layer 2₂₆-4₂ attached or can remove one or the other of the layers 2₂₆-4₁ and 2₂₆-4₂ resulting in the FIG. 27 or the FIG. 28 device, respectively.

In FIG. 29, a top view of another embodiment of a mouth closure 2₂₉ is shown. The closure 2₂₉ includes an air-sealing first layer 2₂₉-3 formed of a first part 2₂₉-3₁ and a second part 2₂₉-3₂. At least the first part 2₂₉-3₁ is over a second adhesive layer (not shown, but like the layer 2-4 in FIG. 4 or FIG. 5). The closure 2₂₉ includes a quick-release member in the form of a penetrable membrane which is the second part 2₂₉-3₂. The second part 2₂₉-3₂ is easily opened with a finger while the first part 2₂₉-3₁ is more durable. When a person, such as person 1 in FIG. 2, has an urgent need for air through the mouth, a finger can be used to easily puncture the second part 2₂₉-3₂ to allow air to pass through the lips 6 and the mouth.

In FIG. 30, a back view of a first embodiment of the mouth closure 2₂₉ of FIG. 29 is shown. The closure 2₂₉ includes an air-sealing first layer 2₂₉-3 and an air-sealing second adhesive layer 2₂₉-4₁ that seals the first layer 2₂₉-3 around and over the lips 6 in a manner similar to FIG. 3. The adhesive layer 2₂₉-4₁ is located around the outer edge of the air-sealing first layer 2₂₉-3 and hence the adhesive layer 2₂₉-4₁, when adhered to a person 1 as shown in FIG. 2, surrounds the lips 6 without substantial contact to the lips 6. The adhesive layer 2₂₉-4₁ is shaped having a width placed around the periphery of the first layer 2₂₉-3 leaving an opening in the center. The first layer 2₂₉-3 includes a quick-release member in the form of the membrane 2₂₉-5.

In FIG. 31, a top view of another embodiment of a mouth closure 2₃₁ is shown. The closure 2₃₁ includes an air-sealing first layer 2₃₁-3 over a second adhesive layer (not shown, but like the layer 2-4 in FIG. 4 or FIG. 5). The closure 2₃₁ includes a quick-release member in the form of a penetrable membrane 2₃₁-5 in a portion of the air-sealing first layer 2₃₁-3 and until penetrated the membrane 2₃₁-5 is air-sealing. When a person, such as person 1 in FIG. 2, has an urgent need for air through the mouth, a finger can be used to easily puncture the membrane 2₃₁-5 to allow air to pass through the lips 6 and the mouth.

In FIG. 32, a back view of a first embodiment of the mouth closure 2₃₁ of FIG. 31 is shown. The closure 2₃₁ includes an air-sealing first layer 2₃₁-3 and an air-sealing second adhesive layer 2₃₁-4₁ that seals the first layer 2₃₁-3 around and over the lips 6 in a manner similar to FIG. 3. The adhesive layer 2₃₁-4₁ is located around the outer edge of the air-sealing first layer 2₃₁-3 and hence the adhesive layer 2₃₁-4₁, when adhered to a person 1 as shown in FIG. 2, surrounds the lips 6 without substantial contact to the lips 6. The adhesive layer 2₃₁-4₁ is shaped having a width placed around the periphery of the first layer 2₃₁-3 leaving an opening in the center. The first layer 2₃₁-3 includes a quick-release member in the form of the membrane 2₃₁-5.

In FIG. 33, a top view of another embodiment of a mouth closure 2₃₃ is shown. The closure 2₃₃ includes an air-sealing first layer 2₃₃-3 over a second adhesive layer (not shown, but like the layer 2-4 in FIG. 4 or FIG. 5). The closure 2₃₃ includes a quick-release member in the form of a penetrable membrane 2₃₃-5 in a portion of the air-sealing first layer 2₃₃-3 and until penetrated the membrane 2₃₃-5 is air-sealing. When a person, such as person 1 in FIG. 2, has an urgent need for air through the mouth, a finger can be used to easily puncture the membrane 2₃₃-5 to allow air to pass through the lips 6 and the mouth.

In FIG. 34, a back view of a first embodiment of the mouth closure 2₃₃ of FIG. 33 is shown. The closure 2₃₃ includes an air-sealing first layer 2₃₃-3 and an air-sealing second adhesive layer 2₃₃-4₁ that seals the first layer 2₃₃-3 around and over the lips 6 in a manner similar to FIG. 3. The adhesive layer 2₃₃-4₁ is located around the outer edge of the air-sealing first layer 2₃₃-3 and hence the adhesive layer 2₃₃-4₁, when adhered to a person 1 as shown in FIG. 2, surrounds the lips 6 without substantial contact to the lips 6. The adhesive layer 2₃₃-4₁ is shaped having a width placed around the periphery of the first layer 2₃₃-3 leaving an opening in the center. The first layer 2₃₃-3 includes a quick-release member in the form of the membrane 2₃₄-5.

In FIG. 31 through FIG. 34, the closure 2₃₁ with the first embodiment adhesive layer 2₃₁-4 and the closure 2₃₃ with the second adhesive layer 2₃₃-4 are examples of closures that have a no overlap of adhesive layer on the skin when used at different times. The adhesive layer 2₃₁-4 is located around the periphery of the first layer 2₃₁-3 and the second adhesive layer 2₃₃-4 fits entirely within the projection of the adhesive layer 2₃₁-4 on the skin. Many different shapes may be employed to form sets of closures that have small or no overlaps so that use of different ones from day to day reduces skin irritation.

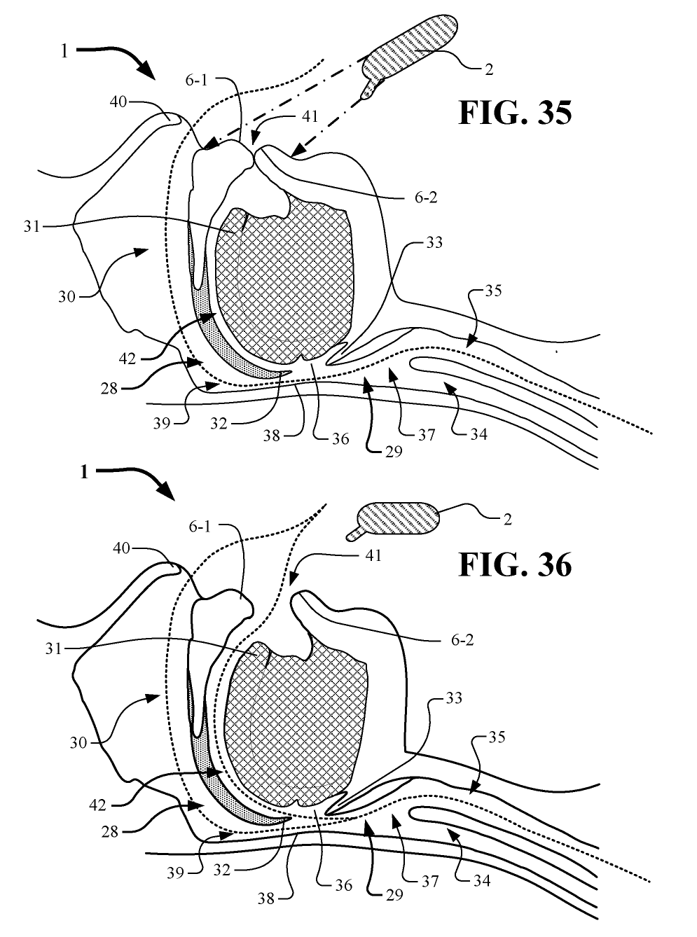

In FIG. 35, a cross-sectional anatomical sagittal view is shown of a person 1 in the back-lying position. The upper lip 6-1 contacts the lower lip 6-2 so that mouth 41 is closed and ready to receive the closure 2.

In FIG. 35, the air path is through the upper air way 28, the lower airway 29 and the lungs. The air path is through the upper airway 28 including the nose 40, the nasal passage 30, the throat 39, the soft palate 32, the posterior throat 38. The air path is through the lower airway 28 including the oropharynx 36, the laryngopharynx 37, the larynx 35 and the lungs (not shown). Because mouth 41 is closed the air flow is not through the mouth passage 42, the tongue 31 and the mouth 41.

In FIG. 36, a cross-sectional anatomical sagittal view is shown of a person 1 in the back-lying position with unobstructed breathing. The upper lip 6-1 is separated from the lower lip 6-2 so that mouth 41 is open. Since the mouth 41 is open, the person 1 is not ready to receive the closure 2.

In FIG. 36, the air path is through the upper air way 28, the lower airway 29 and the lungs. The air path is through the nose 40, the nasal passage 30, to the throat 39, along the soft palate 32, along the posterior throat 38, the oropharynx 36, the laryngopharynx 37, the larynx 35 and to the lungs (not shown). Because mouth 41 is open, the air flow is also through the mouth passage 42, along the tongue 31 and the mouth 41.

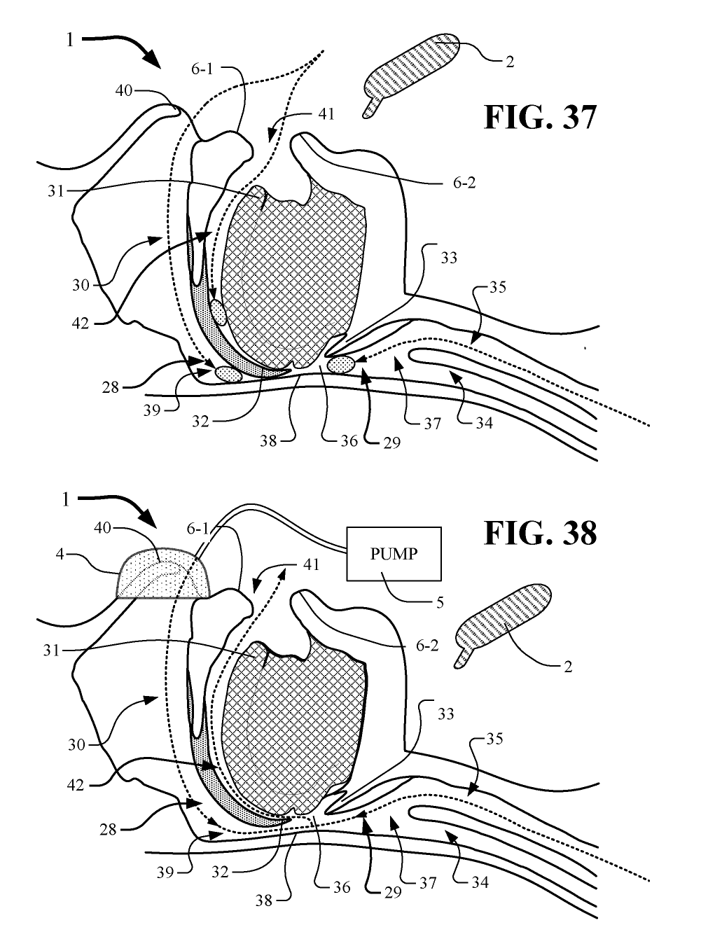

In FIG. 37, a cross-sectional anatomical sagittal view is shown of a person 1 in the back-lying position with obstructed breathing. The upper lip 6-1 is separated from the lower lip 6-2 so that mouth 41 is open. Since the mouth 41 is open, the person 1 is not ready to receive the closure 2.

In FIG. 37, the air path is attempted to be through the upper air way 28. The air path is through the nose 40, the nasal passage 30, to the throat 39. An obstruction is encountered, for example, the soft palate 35 is against the posterior throat wall 38 and air flow is otherwise blocked along the soft palate 32 and the posterior throat 38 so that air does not pass along the lower airway 29 including the oropharynx 36, the laryngopharynx 37, the larynx 35 and the lungs (not shown). Although the mouth 41 is open, the air flow also encounters blockage in the mouth passage 42 so that air does not flow along the tongue 31 and mouth 41.

In FIG. 38, a cross-sectional anatomical sagittal view is shown of a person 1 in the back-lying position with Positive Airway Pressure assisted breathing. The upper lip 6-1 is separated from the lower lip 6-2 so that mouth 41 is open. Since the mouth 41 is open, the person 1 is not ready to receive the closure 2. A positive pressure nose mask 4 is affixed over the nose 40 to control the Positive Airway Pressure assisted breathing.

In FIG. 38, the air path is through the upper air way 28 and the lower airway 29. The air path includes the pump 5 and mask 4, the nose 40, the nasal passage 30, the throat 39, along the soft palate 32, along the posterior throat 38, the oropharynx 36, the laryngopharynx 37, the larynx 35 and the lungs (not shown). Because mouth 41 is open, the air flow is also through the mouth passage 42, along the tongue 31 and the mouth 41.

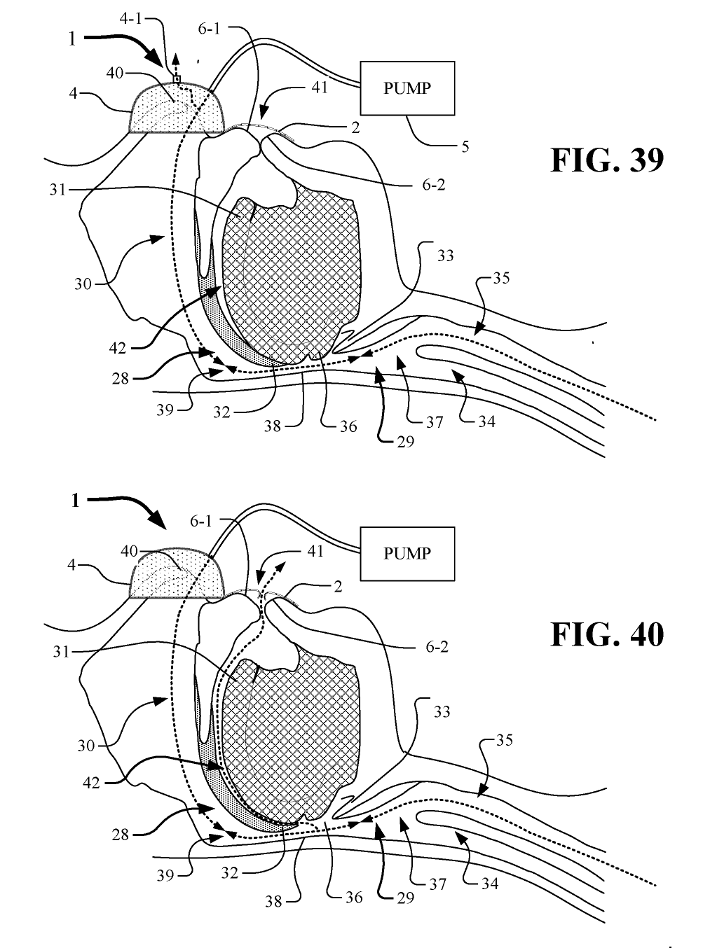

In FIG. 39, a cross-sectional anatomical sagittal view is shown of a person 1 in the back-lying position with Positive Airway Pressure assisted breathing. The upper lip 6-1 is in contact with the lower lip 6-2 so that mouth 41 is closed. With mouth 41 closed, the person 1 has the closure 2 affixed over the lips 6, including upper lip 6-1 and lower lip 6-2, and to adjacent skin region to seal the mouth 41. A positive pressure nose mask 4 is affixed over the nose 40 to control the Positive Airway Pressure assisted breathing without unwanted leakage through the mouth 41. The closure 2 is any of the closures previously described and equivalents thereof.

In FIG. 39, the air path is through the upper airway 28 and the lower airway 29. The air path includes the pump 5 and mask 4, the nose 40, the nasal passage 30, the throat 39, along the soft palate 32, along the posterior throat 38, the oropharynx 36, the laryngopharynx 37, the larynx 35 and the lungs (not shown). Because mouth 41 is closed and sealed by closure 2, no leakage air flow occurs through the mouth passage 42, along the tongue 31 and the mouth 41.

In FIG. 39, the anti-apnea mouth closure 2 contributes to the ideal operation controlling the upper airway 28 to be in a “pneumatic splint” state through the application of continuous positive airway pressure from the pump 5 and nose mask 4. The air flow into the nose mask 4 through the input port from pump 5 equals the air flow out of the nose mask 4 both through the nose mask output port 4-1 (to ambient atmosphere) and through the upper airway 28 and lower airway 29 connected to the lungs. During inspiration, some of the input air flow through the input port from pump 5 exits the nose mask 4 as the inspiration airflow into the upper airway 28 and lower airway 29 to the lungs while the remainder of the input air flow exits the mask through the mask output port. During expiration operation, airflow from the lungs to the lower airway 29 and upper airway 28 enters into the nose mask 4, and together with airflow from the input port connected to pump 5 together exit the nose mask 4 through the nose mask output port 4-1. With such operation and when an outward air flow (to ambient) through the nose mask output port 4-1 is maintained throughout the respiratory cycle, a positive air pressure in the nose mask 4 and in the upper airway 28 is maintained so as to establish the “pneumatic splint” condition that holds the upper airway 28 open throughout the respiratory cycle.

In FIG. 40, a cross-sectional anatomical sagittal view is shown of a person 1 in the back-lying position with Positive Airway Pressure assisted breathing. The upper lip 6-1 is in contact with the lower lip 6-2 so that mouth 41 is closed. With mouth 41 closed, the person 1 has the mouth closure 2 affixed over the lips 6, including upper lip 6-1 and lower lip 6-2, and to adjacent skin region to seal the mouth 41. A positive pressure nose mask 4 is affixed over the nose 40 to control the Positive Airway Pressure. The mouth closure 2 has been penetrated to break the seal over the mouth so the air flow occurs through the mouth 41.

In FIG. 40, the air path is through the upper air way. The air path includes the pump 5 and mask 4, the nose 40, the nasal passage 30, the throat 39, along the soft palate 32, along the posterior throat 38, the oropharynx 36, the laryngopharynx 37, the larynx 35 and the lungs (not shown). Because the closure 2 over the mouth 41 has been ruptured and the mouth 41 is somewhat open, the air flow is also through the mouth passage 42, along the tongue 31 and the mouth 41.

In the present specification including claims, the term “quick-release member” means any member which can be used to quickly and easily break the closure seal over the mouth and allow breathing through the mouth. As previously described, the quick-release member is a tab, such as tab 2-1 in FIG. 3, is a penetrable membrane, such as membrane 27-5 in FIG. 7 and can be any other member that breaks the seal over the mouth. For example, the quick-release member includes the rip section 2₂₃-1 of FIG. 23 which tears the rip section 2₂₃-1′ to break the seal and includes a weaker layer or part, such as part 2₂₉-3₂ of FIG. 29, which can be penetrated by a finger, by forceful opening of the mouth or by a forceful inhalation.

In the present specification including claims, the term “air-sealing” means preventing or limiting air leak through the mouth during Positive Airway Pressure treatment of Obstructive Sleep Apnea. The term “air-sealing” can range from complete air blockage to partial air flow where the amount of air that passes through the closure is sufficient to facilitate Positive Airway Pressure treatment. The amount of air that passes through the closure, if any, is controlled by leak holes or other structure provided in the air-sealing layers or by the air permeability of the materials form the closure.

In the present specification including claims, the term “adhesive” means a material that adheres the closure to the skin in a manner that facilitates formation of an air seal. In some embodiments, the adhesive member is a gel material. This gel material is “tacky” so that it grips the skin sufficiently for air-sealing of the closure over the lips and mouth during Positive Airway Pressure treatment. A gel material typically is reusable before losing its tackiness. A gel can be used together with head gear and headgear/straps to hold the closure and gel adhesive in place.

In the present specification including claims, the term “CPAP” means any positive airway pressure (PAP) device such as BiPAP, AutoPAP, etc., used to maintain adequate airway patency and function.

In the present specification including claims, the term “Obstructive Sleep Apnea” means any condition for which positive airway pressure is utilized such as Central Sleep Apnea, Mixed Sleep Apnea, Complex Sleep Apnea, etc.

In the present specification including claims, the term “closure” includes any member which functions to limit or block the flow of air through the mouth and lips and at times is referred to as a tape, mask, cover or strip.

While the invention has been particularly shown and described with reference to preferred embodiments thereof it will be understood by those skilled in the art that various changes in form and details may be made therein without departing from the scope of the invention.

1. A closure for use during Positive Airway Pressure treatment of Obstructive Sleep Apnea for controlling air leakage through lips and mouth of a person comprising:
a first member formed of an air-sealing layer for extending over the lips of and to an adjacent region on skin of the person,
a second member formed of an adhesive layer adhered to the first member for placement on at least the adjacent region on the skin where the adhesive layer contacts the skin for adhesively adhering the second member to the skin in the adjacent region to form an air seal over the mouth,
the first member and second member covering the whole mouth without any uncovered regions to prevent air leakage through the mouth,
a quick-release member for breaking the air seal.

2. The closure of claim 1 wherein the quick-release member is a tab attached to the first member.

3. The closure of claim 1 wherein the quick-release member is permeable member within a central region of the first member.

4. The closure of claim 1 wherein the quick-release member is a central region of the first member where the central region is substantially thinner than other regions of the first member.

5. The closure of claim 1 wherein the quick-release member is a central region of the first member where the central region is perforated.

6. The closure of claim 1 wherein the first member includes a chin strap.

7. The closure of claim 1 wherein the first member has a periphery in the adjacent region and wherein the second member is located around the periphery of the first member with an opening in a center region so as not to contact the lips.

8. The closure of claim 1 wherein the first member has a periphery in the adjacent region and wherein the second member includes a first part located around the periphery of the first member and with a second part located within the first part where the second part has an opening in a center region so as not to contact the lips.

9. The closure of claim 1 wherein the first member has a periphery in the adjacent region and wherein the second member includes a first part located around the periphery of the first member and with a second part located within the first part where the second part has an opening in a center region so as not to contact the lips and wherein at least one the first part and the second part are removable to provide the closure with only the first part or the second part.

10. The closure of claim 1 wherein the first member has a periphery in the adjacent region and wherein the second member includes a part located within a first region located around the periphery of the first member where the second part has an opening in a center region so as not to contact the lips.

11. A closure for use during Positive Airway Pressure treatment of Obstructive Sleep Apnea for controlling air leakage through lips and mouth of a person, said closure being one of a set of closures where the closure comprises:
a first member formed of an air-sealing layer for extending over the lips of and to an adjacent region on the skin of the person,
a second member formed of an adhesive layer adhered to the first member for placement on at least the adjacent region on the skin where the adhesive layer contacts the skin for adhesively adhering the second member to the skin to form an air seal over the mouth,
the first member and second member covering the whole mouth without any uncovered regions to prevent air leakage through the mouth,
a quick-release member for breaking the air seal,
and where the set of closures replicates the closure with the second member for the closure replicated in the set of closures and arrayed in the set of closures such that the placement on the skin occurs at different portions of the skin to reduce or eliminate overlap of adhesive whereby the set of closures tend to reduce agitation of the skin.

12. The closure of claim 11 wherein the quick-release member is a tab attached to the first member.

13. The closure of claim 12 wherein the quick-release member is permeable member within a central region of the first member.

14. The closure of claim 12 wherein the quick-release member is a central region of the first member where the central region is substantially thinner than other regions of the first member.

15. The closure of claim 12 wherein the quick-release member is a permeable member within a central region of the first member.

16. The closure of claim 12 wherein the second member is located around the periphery of the first member with an opening in the center so as not to contact the lips.

17. The closure of claim 12 wherein the first member has a periphery in the adjacent region and wherein the second member includes a first part located around the periphery of the first member and with a second part located within the first part where the second part has an opening in a center region so as not to contact the lips.

18. The closure of claim 12 wherein the first member has a periphery in the adjacent region and wherein the second member includes a first part located around the periphery of the first member and with a second part located within the first part where the second part has an opening in a center region so as not to contact the lips and wherein at least one the first part and the second part are removable to provide the closure with only the first part or the second part.

19. The closure of claim 12 wherein the first member has a periphery in the adjacent region and wherein the second member includes a part located within a first region located around the periphery of the first member where the second part has an opening in a center region so as not to contact the lips.

20. A method of Positive Airway Pressure treatment of Obstructive Sleep Apnea by controlling air through a nose and controlling air leakage through lips and mouth of a person comprising:
applying a positive air pressure through a nose mask over the nose,
fixing a mouth closure over the lips and mouth to form a seal for controlling air leakage through the lips and mouth where the mouth closure includes,
a first member formed of an air-sealing layer for extending over the lips of and to an adjacent region on skin of the person,
a second member formed of an adhesive layer adhered to the first member for placement on at least the adjacent region on the skin where the adhesive layer contacts the skin for adhesively adhering the second member to the skin to form an air seal over the mouth,
the first member and second member covering the whole mouth without any uncovered regions to prevent air leakage through the mouth,
a quick-release member for breaking the air seal.
